(19) United States
(12) Patent Application Publication
(10) Pub. No.: US 2019/0070234 A1
(43) Pub. Date: Mar. 7, 2019

(54) METHOD FOR FOODBORNE PATHOGEN REDUCTION IN POULTRY

(71) Applicant: CAN TECHNOLOGIES, INC., Wayzata, MN (US)

(72) Inventors: Donald McINTYRE, Pageland, SC (US); Michael MITCHELL, Richmond, KY (US)

(73) Assignee: CAN TECHNOLOGIES, INC., Wayzata, MN (US)

(21) Appl. No.: 16/182,035

(22) Filed: Nov. 6, 2018

Related U.S. Application Data
(63) Continuation of application No. 15/000,835, filed on Jan. 19, 2016.
(60) Provisional application No. 62/104,530, filed on Jan. 16, 2015.

(51) Int. Cl. A61K 36/064; A23K 10/12; A61K 33/06; A61K 9/00; A23K 50/75; A23K 10/16; A61K 33/42; A61K 33/00

(52) U.S. Cl. CPC A61K 36/064; A23K 10/12; A61K 33/06; A61K 9/0056; A23K 50/75; A61K 2300/00; A61K 33/42; A61K 33/00; A61K 2236/19; A23K 10/16

(57) ABSTRACT

A method for introducing a fermented product to poultry to reduce foodborne pathogens. The fermented product contains functional metabolites produced by Saccharomyces cerevisiae and is added at a particular ratio to the feed for the most effective results.

### CROSS-REFERENCE TO RELATED APPLICATIONS

The present application claims priority to provisional patent application 62/104,530 which was filed on Jan. 16, 2015, and is hereby expressly incorporated by reference in its entirety.

### BACKGROUND OF THE INVENTION

Food safety has become a priority for consumers, food producers as well as for federal and state regulatory agencies. Significant changes in federal food safety and feed production regulations have occurred with the passage of the U. S. Food Safety and Modernization Act. Activity at the state level has included the passage of labeling laws and animal care legislation.

Despite significant progress by the poultry and red meat industries in reducing foodborne pathogens, human illness due to consumption of these products persists. Pathogenic bacteria such as Salmonella, Campylobacter, and Escherichia coli are frequently associated with consumption of animal protein products, and are often cited among the top five pathogens causing foodborne illness in the U.S. Companies producing foods of animal origin must implement effective pre-harvest food safety programs in order to lower the risk of human illness.

Responsibility for foodborne pathogen control (e.g., Salmonella, Campylobacter, etc.) lies within each segment of the food chain, from farm to fork, in order to produce a safe, sustainable, and abundant food supply. Research-proven technologies must be key components in a company's food safety plans.

Most efforts to reduce pathogen numbers in food animals and products, including chemicals applied during processing, are not completely effective. Research shows that the pre-harvest pathogen contamination of animals, especially poultry, correlates directly with the post-harvest presence of pathogens found in the plant (Berghaus, R., S. G Thayer, B. F. Law, R. M. Mild, C. L. Hofacre and R. S. Singer, 2013, Enumeration of Salmonella and Campylobacter in environmental farm samples and processing plant carcass rinses from commercial broiler chicken flocks and in food product, Appl. Environ. Microbiol. 1-37.) (Pearson, A. D., M. Greenwood, T. D. Healing, D. Rollins, M. Shahamat, J. Donaldson, and R. R. Colwell. 1993. Colonization of Broiler Chickens by Waterborne Campylobacter jejuni. Applied and Environmental Microbiology. April 1993, p. 987-996.); (Corry, J. E. L. and H. I. Atabay. 2001. Poultry as a source of Campylobacter and related organisms. Journal of Applied Microbiology 90: 96S-114S.); (Corry, J. E. L., V. M. Allen, W. R. Hudson, M. F. Breslin and R. H. Davies. 2002. Sources of salmonella on broiler carcasses during transportation and processing: modes of contamination and methods of control. Journal of Applied Microbiology 92: 424-432.); (Heyndrickx, M., D. Vandekerchove, L. Herman, I. Rollier, K. Grijspeerdt and L. De Zutter. 2002. Routes for salmonella contamination of poultry meat: Epidemiological study from hatchery to the slaughterhouse. Epidemiology and Infection. 129 (2): 253-265.); (Rosenquist, H., H. M. Sommer, N. L. Nielsen and B. B. Christensen. 2006. The effect of slaughter operations on the contamination of chicken carcasses with thermotolerant Campylobacter. International Journal of Food Microbiology. 108 (2): 226-232.); (Johannessen, G. S., G. Johnsen, M. Økland, K. S. Cudjoe and M. Hofshagen. 2007. Enumeration of thermotolerant Campylobacter spp. from poultry carcasses at the end of the slaughter-line. Letters in Applied Microbiology. 44: 92-97.; Hue, O., V. Allaina, M. Laisneya, S. Le Bouquina, F. Lalandea, I. Petetina, S. Rouxela, S. Quesnea, P. Gloaguena, M. Picherota, J. Santolinia, S. Bougearda, G. Salvata, and M. Chemalya. 2011. Campylobacter contamination of broiler caeca and carcasses at the slaughterhouse and correlation with Salmonella contamination. Food Microbiology. 28 (5): 862-868.); Cox, L. A. and R. S. Singer. 2012. Confusion over Antibiotic Resistance: Ecological Correlation Is Not Evidence of Causation. Foodborne Pathogens and Disease. 9(8): 776-786.).

Pre-harvest interventions have been studied previously but have been largely ineffective, or have demonstrated inconsistent success at best. These early interventions include: a) vaccination of breeding stock to help reduce pathogen load in the progeny, b) vaccinating commercial meat birds to reduce pathogen load at market age, c) use of probiotics or competitive exclusion live organisms composed of less harmful bacteria, d) adding organic acids to the feed in an effort to reduce pH in the gastrointestinal (GI) tract and hopefully suppress food borne pathogens in the crop or gut of the bird/animal, e) including pre-biotic type compounds to the feed to prevent pathogen colonization inside the bird, and f) the addition of essential oils like oregano, cinnamon, ginger or rosemary that may have an impact on pathogen levels in the intestine of poultry.

The use of vaccines, for example, requires an adequate dose to be administered effectively to every bird/animal in the flock/herd. Each individual must be physically able to recognize the vaccine strain and to mount the desired protection against that pathogen. This response is normally distributed within a flock such that some animals achieve good protection while others do not, leaving compromised individuals at risk for invasion by natural pathogens. Most vaccines are not universally successful and might only protect against specific serotypes present within the flock or environment. The use of vaccines, while helpful, has not been entirely successful and must be administered effectively with an associated expense and often with detrimental effects on performance of the animal due to handling and negative reactions to the vaccination.

Due to the nature of digestion in birds, where hydrochloric acid is secreted in the stomach and a natural pH of 2 is normal, the addition of more acid to the water or feed is highly unpalatable to birds and completely unable to influence natural digestion. Additionally, in birds, when feed passes into the small intestine the pH must be increased to approximately 6.4 to accommodate optimal nutrient absorption. Anything less requires the bird to secrete more sodium bicarbonate to buffer the slurry or suffer harmful effects and reduced nutrient absorption and slower growth. Endeavoring to overcome this shortfall, some have tried to produce a coated acid to attempt to pass into the lower intestine where it can be more effective. Once again, highly variable results have been seen with this premise due to the inability to control conditions within the gut that consistently deliver the correct amount of product at the right time to produce the desired outcome.

While there is no “silver bullet” to completely eliminate foodborne pathogens, a multi-step food safety program, including pre-harvest mitigation at the farm level, has been shown to be most effective.

It is an object of the invention to provide a pre-harvest process for reducing foodborne pathogens in poultry.

It is also an object of the invention to provide a process for reducing foodborne pathogens utilizing a fermented nutritional health product.

It is a further object of the invention to provide a process for reducing foodborne pathogens that do not interfere with the digestive process of poultry.

### SUMMARY OF THE INVENTION

A fermented product, such as Diamond V's Original XPC, is fed at particular rates such that the product which contains functional metabolites effectively and safely arrives to the target location within the GI tract of poultry, without being altered or compromised. The functional metabolites and particular ratios used in feeding poultry facilitate the desired effect on the poultry's immune response and reduce pathogens. This process reduces the influence of normal variation, naturally seen in gut microbiota and enteric conditions that reduce the consistency and overall efficacy of previously tried solutions.

The net result of feeding Diamond V's fermented products to poultry can be seen in the animal's ability to protect itself naturally by causing the release of natural cytokines. These messengers trigger a cascade of events measurable in the tissues, organs, and the blood that result in a healthier animal and one prepared to face unanticipated challenges and stress, by many different disease or environmental factors. This fermented product can be included in the finished feed or in the premix with vitamins and trace minerals. These stable compounds arrive at the appropriate site within the digestive tract in the necessary ratio to produce desired results.

### DETAILED DESCRIPTION

The inventive process introduces a fermented product to the feed of poultry which results in less foodborne pathogens. The optimal dose may vary depending on the particular animal to be treated. The fermented product is preferably derived from a natural media that has been treated with at least one type of yeast. The yeast at least partially consume the media, and due to certain chemical reactions, produce functional metabolites. The fermented product is then added to the feed to achieve a particular ratio of fermented product to total feed. The preferred range is the fermented product comprising between 0.004% and 0.2% of the total feed, with an ideal percentage of 0.125%.

Original XPC from Diamond V (hereinafter “Original XPC”) contains functional metabolites produced during a fermentation process by the yeast Saccharomyces cerevisiae. The Original XPC can be utilized as the fermented product in the process described herein. This fermented product when added to the feed of poultry has been shown to reduce the colonization and shedding of pre-harvest foodborne pathogens. Original XPC falls under “yeast culture” in the 2015 Official Publication of the Association of American Feed Control Officials. As Original XPC is the preferred fermented product, the ideal characteristics of the fermented product include: crude protein is a minimum of 12.0%, crude fat is a minimum of 1.2%, crude fiber is a maximum of 30.0%, and ash is a maximum of 10.8%, together with at least fourteen amino acids at varying levels greater than 0.16% such as leucine, proline, glycine, and valine, and also a diverse group of minerals that includes potassium, phosphorus, and calcium.

Testing has demonstrated that feeding Original XPC to egg laying hens, meat-type chickens and turkeys will reduce the prevalence (% positives) of salmonella and campylobacter isolated from birds and their environment. Results from this research also show a reduction in the number of salmonella and campylobacter organisms isolated from the reduced number of positive birds. Testing has shown that a recommended feeding rate of 2.5 pounds of fermented product per ton of total feed has the most beneficial effects of reducing pathogens related to poultry. The effective range in poultry can vary, however, the inventive process has a range of fermented product between 0.004% and 0.2% of the complete diet, as fed. The most effective and economical dose for poultry is the fermented product comprising 0.125% of the total feed, which corresponds to the 2.5 pounds of fermented product per ton of total feed.

Studies were implemented to determine the most effective feeding rates of the fermented product, specifically Original XPC, to be used in the process. The primary objectives of a first study were to determine: 1) if 2.5 pounds of fermented product, specifically of Original XPC, per ton of total feed would decrease the pathogen, Salmonella Typhimurium (hereinafter “S.T.”), transmission from pen mates challenged at one day of age, thereby preventing colonization and shedding throughout a hen poults life to processing age; and 2) the effectiveness of the fermented product, specifically of Original XPC, in prevention or reduction of a Salmonella Heidelberg (hereinafter “S.H.”) colonization and/or shedding between pen mate hens (exposed at 56 days of age) until processing age of 84 days.

The results of the first study indicate that while a fermented product level, specifically an Original XPC level of 2.5 pounds per ton of total feed did not significantly reduce S.T. or S.H. prevalence in the environment or ceca of poults at either 42 day or 84 day sampling, it did reduce the load. Salmonella Heidelberg levels in the ceca on day 84 was 0.42 logs lower in Original XPC treated poults, equating to a 61% reduction in S.H. levels in the ceca of Original XPC treated hens at processing age. A major concern of turkey processors is the small number of hens with extremely high Salmonella levels in their ceca. These higher colonized hens can result in further contamination of processing equipment contamination, which will lead to an increased risk of cross-contamination. The results indicate that a fermented product, specifically Original XPC, is effective at lowering mean Salmonella counts at a particular amount or feed rate. However, is the lower number of Original XPC treated birds with extremely high S.H. levels in their ceca is a more significant finding for the processing plant. It was observed some non Original XPC treated hens had S.H. counts greater than 10,000 S.H. cells per gram of ceca content.

A second study compared cecal Salmonella most probable numbers (hereinafter “MPN”) and prevalences between treatment groups and challenge status categories using generalized estimating equations (hereinafter “GEE”) linear and logistic models, respectively, to account for the correlation between responses of birds from the same pens. These models were estimated using robust standard errors and an exchangeable working correlation structure. For the comparison of Salmonella MPN, samples with a negative culture result by the MPN method but with a positive culture result by primary or secondary enrichment, were arbitrarily assigned an MPN value equal to one half the minimum detection limit of the MPN assay. MPN values were log-transformed prior to statistical analysis. All statistical testing assumed a two-sided alternative hypothesis, and P<0.05 was considered significant.

The results of the second study are summarized in Tables 1 and 2 below by treatment group and bird challenge status (dosed or not dosed). The pen-level distribution of Salmonella prevalences is illustrated in Table 1. In the univariate analysis, there was a significant difference between treatment groups with respect to Salmonella prevalence (P=0.005); the prevalence in the Original XPC 2.5 pounds per ton group was significantly lower than in the control group, while the prevalence in the Original XPC 1.25 pounds per ton group was intermediate and not significantly different from the other two groups. Compared to birds receiving the control treatment, the chance of Salmonella detection was 20% lower in birds receiving the Original XPC at 1.25 pounds per ton of total feed treatment [odds ratio (95% CI)=0.80 (0.26, 2.4)] and 66% lower in birds receiving the Original XPC at 2.5 pounds per ton of total feed treatment [odds ratio (95% CI)=0.34 (0.16, 0.75)].

TABLE 1
Salmonella prevalences in ceca collected from 15 birds per pen in 8 pens
for each of 3 treatments. Of the 15 birds sampled in each pen, 5 were
individually dosed with Salmonella at 1 day of age and 10 were
not individually dosed with Salmonella.

| Variable | n | No. positive (%) | ^{†}P |
|---|---|---|---|
| Treatment | | | 0.005 |
| Control | 120 | 46 (38.3)^{a} | |
| XPC 1.25 lb/t | 120 | 40 (33.3)^{a,b} | |
| XPC 2.5 lb/t | 120 | 21 (17.5)^{b} | |
| Dosed with Salmonella | | | 0.647 |
| No | 240 | 73 (30.4)^{a} | |
| Yes | 120 | 34 (28.3)^{a} | |

^{†}Univariate generalized estimating equations logistic model adjusted for clustering by pen. For treatment and for dosed, prevalences with a superscript in common do not differ with a level of significance of 5% over all comparisons.

TABLE 2
Geometric mean (95% CI) Salmonella MPN per gram in culture-positive ceca samples
by treatment group and challenge status (dosed or not dosed).

| Dosed | Control | XPC 1.25 lb/t | XPC 2.5 lb/t | Total |
|---|---|---|---|---|
| No | 1.8 (1.0, 3.0) | 10 (1.3, 81) | 1.2 (0.64, 2.4) | 2.7^{a} (1.3, 5.5) |
| Yes | 5.3 (1.0, 27) | 14 (2.1, 98) | 4.0 (0.32, 51) | 8.2^{a} (2.6, 26) |
| Total | 2.8^{a.b} (1.3, 6.1) | 12^{b} (3.1, 50) | 1.7^{a} (0.74, 4.0) | 4.4 (2.3, 8.6) |

Within rows and columns, marginal means with a superscript in common do not differ with a level of significance of 5% over all comparisons. Sample sizes: control not dosed, n = 19; control dosed, n = 14;
XPC 1.25 lb/t not dosed, n = 12; XPC 1.25 lb/t dosed, n = 16.; XPC 2.5 lb/t not dosed, n = 10; XPC 2.5 lb/t dosed, n = 4.

Other studies utilizing a control group and an experimental group receiving Original XPC at a rate of 2.5 pounds per ton of total feed produced similar results. Specifically, those results demonstrated that poultry fed the Original XPC had a significantly lower Salmonella prevalence compared to the control group.

Having thus described the invention in connection with the several embodiments thereof, it will be evident to those skilled in the art that various revisions can be made to the several embodiments described herein with out departing from the spirit and scope of the invention. It is our intention, however, that all such revisions and modifications that are evident to those skilled in the art will be included with in the scope of the following claims. Any elements of any embodiments disclosed herein can be used in combination with any elements of other embodiments disclosed herein in any manner to create different embodiments.

1-13. (canceled)

14. A method for improving weight gain in poultry which have been administered a coccidia vaccine, comprising the steps of:
supplementing a feed with a fermentation product derived from a yeast to produce a total feed,
wherein the fermentation product is derived at least partially from Saccharomyces cerevisiae; and
wherein the fermentation product is added to the feed such that the fermentation product is between 0.004% and 0.2% of the weight of the total feed,
and feeding the total feed to poultry, wherein the poultry have been administered a coccidia vaccination prior to feeding,
wherein the body weight of the poultry after 16 days is greater than the body weight of control poultry which were administered a coccidia vaccine but not fed a total feed including the fermentation product.

15. The method of claim 14, wherein:
the fermentation product comprises functional metabolites produced by a Saccharomyces cerevisiae yeast.

16. The method of claim 14, wherein:
the fermentation product has a crude protein content, a crude fat content, and a crude fiber content; and comprises
amino acids,
potassium, phosphorous and calcium.

17-18. (canceled)

19. The method of claim 16, wherein the crude protein content is a minimum of 12.0%.

20. The method of claim 16, wherein the crude fat content is a minimum of 1.2%.

21. The method of claim 16, wherein the crude fiber content is a maximum of 30.0%.

22. The method of claim 16, wherein the ash content of the fermentation product is a maximum of 10.8%.

23. The method of claim 14, wherein the amount of fermentation product added to the feed is about 0.125% of the total feed.

24. The method of claim 14, wherein the poultry is not administered an antibiotic after coccidia vaccination.

25. The method of claim 14, wherein the increase in body weight of the poultry after 16 days is at least 5% compared to the control poultry.

26. The method of claim 14, wherein the increase in body weight of the poultry after 28 days is at least 4% compared to the control poultry.

27. The method of claim 14, wherein the feed conversion ratio (FCR) of the poultry is improved compared to the control poultry.

28. The method of claim 14, wherein the feed intake of the poultry is improved compared to the control poultry.
